(19) United States
(12) Patent Application Publication
(10) Pub. No.: US 2026/0224383 A1
(43) Pub. Date: Aug. 6, 2026

(54) KNEE BRACES AND METHODS OF MAKING AND USING THE SAME

(71) Applicant: Joshua Robert MINER, Ashburn, VA (US)

(72) Inventor: Joshua Robert MINER, Ashburn, VA (US)

(21) Appl. No.: 19/380,967

(22) Filed: Nov. 6, 2025

Related U.S. Application Data
(60) Provisional application No. 63/716,967, filed on Nov. 6, 2024.

(51) Int. Cl. A61F 5/01

(52) U.S. Cl. CPC A61F 5/0123; A61F 2005/0137; A61F 2005/0174

(57) ABSTRACT

The disclosure is directed to knee braces and methods of making and using the same. A knee brace configured to be attached to a leg of a user, the knee brace comprising: (A) a left side assembly including: a left upper cheek; a left lower cheek; a left offset hinge that rotatably connects the left upper cheek to the left lower cheek, so as to be hinged about a first hinge axis; (B) a right side assembly including: a right upper cheek; a right lower cheek; a right offset hinge that rotatably connects the right upper cheek to the right lower cheek, so as to be hinged about the first hinge axis; (C) a first upper strap; (D) a second upper strap, (E) a third lower strap; and (F) a fourth lower strap.

### RELATED APPLICATION AND PRIORITY

This application claims priority to U.S. Provisional Patent Application 63/716,967 filed Nov. 6, 2024, the content of which is incorporated herein by reference in its entirety.

### BACKGROUND OF THE DISCLOSURE

The disclosure is directed to and relates to knee braces and methods of making and using the same.

Injury to the knee, whether due to sports activities, trauma, or degenerative conditions, often results in significant instability and pain that requires medical intervention. For effective recovery, immobilization or controlled movement of the knee joint can be critical to promote healing of damage, including tissue, ligaments, tendons, cartilage, bone, nerves, membranes, joint capsules, bursae, fat pads and/or blood vessels. Hinged knee braces have been developed to address this need by providing both support and mobility control to the knee.

Traditional hinged knee braces commonly feature a metal or plastic hinge positioned on either side of the knee joint. These hinges allow the brace to lock the knee into a fixed range of motion or position, limiting motion and thereby stabilizing the joint. By controlling movement, the brace functions similarly to a cast used for healing broken bones, ensuring the inner tissues, such as the anterior cruciate ligament (ACL) or meniscus, can properly heal without unnecessary strain. The brace's structure aims to alleviate knee instability, reduce pain, and expedite the recovery process following injury or surgery. Yet, despite their widespread use, many existing, known hinged knee braces have many limitations.

### BRIEF SUMMARY OF THE DISCLOSURE

The disclosure is directed to knee braces and methods of making and using the same. A knee brace configured to be attached to a leg of a user, the knee brace comprising: (A) a left side assembly, the left side assembly including: a left upper cheek configured to engage with an upper leg of the user; a left lower cheek configured to engage with a lower leg of the user; a left offset hinge that rotatably connects the left upper cheek to the left lower cheek, so as to be hinged about a first hinge axis; (B) a right side assembly, the right side assembly including: a right upper cheek configured to engage with the upper leg of the user; a right lower cheek configured to engage with the lower leg of the user; a right offset hinge that rotatably connects the right upper cheek to the right lower cheek, so as to be hinged about the first hinge axis; (C) a first upper strap connected to the left upper cheek and the right upper cheek, the first upper strap configured to encircle the upper leg of the user; (D) a second upper strap connected to the left upper cheek and the right upper cheek, the second upper strap configured to encircle the upper leg of the user, (E) a third lower strap connected to the left lower cheek and the right lower cheek, the third lower strap configured to encircle the lower leg of the user; (F) a fourth lower strap connected to the left lower cheek and the right lower cheek, the fourth lower strap configured to encircle the lower leg of the user.

### BRIEF DESCRIPTION OF THE DRAWINGS

The present disclosure can be more fully understood by reading the following detailed description together with the accompanying drawings, in which like reference indicators are used to designate like or similar elements, and in which:

FIG. 1 is a back perspective view of a knee brace, in accordance with principles of the disclosure.

FIG. 2 is side view of a knee brace, the same or similar to the knee brace of FIG. 1, mounted on a leg of a user, in accordance with principles of the disclosure.

FIG. 2A is schematic side view a knee brace, the same as or similar to the knee brace of FIG. 2, mounted on a leg of a user, in accordance with principles of the disclosure.

FIG. 3 is a side view of a side assembly 20 the same as or similar to the side assembly of the knee brace of FIG. 1, in accordance with principles of the disclosure.

FIG. 4 is a partial side view of the side assembly of FIG. 3, in accordance with principles of the disclosure.

FIG. 5 is a flow schematic state diagram showing different states, i.e. different operational dispositions of a side assembly of a knee brace the same as or similar to the knee brace of FIG. 1, in accordance with principles of the disclosure.

FIG. 6 is a schematic diagram of opposing side assemblies, the same as or similar to the side assemblies of FIG. 1, without straps, in accordance with principles of the disclosure.

FIG. 7 is a side view of a connector assembly, in accordance with principles of the disclosure.

FIG. 8 is a front view of the connector assembly of FIG. 7, in accordance with principles of the disclosure.

FIG. 9 is a side view of a plate, without pins, of a connector assembly of FIG. 7, in accordance with principles of the disclosure.

FIG. 10 is an exploded view of a pin or pin assembly, in accordance with principles of the disclosure.

FIG. 11 is a side view of a washer the same as or similar to the washer of FIG. 8, in accordance with principles of the disclosure.

### DETAILED DESCRIPTION OF THE DISCLOSURE

A few inventive aspects of the disclosed embodiments are explained in detail below with reference to the various figures. Exemplary embodiments are described to illustrate the disclosed subject matter, not to limit its scope, which is defined by the claims. Those of ordinary skill in the art will recognize a number of equivalent variations of the various features provided in the description that follows.

As used herein, any term in the singular may be interpreted to be in the plural, and alternatively, any term in the plural may be interpreted to be in the singular.

Traditional treatments for knee issues, such as surgery, physical therapy, and conventional braces, often fall short due to the knee's limited blood flow, leaving many cases unresolved. The new knee brace of the disclosure takes a novel and different approach in its design, function, and application. The knee brace of the disclosure focuses on restoring functionality by reducing stress on the knee, rather than trying to promote healing directly.

What sets the knee brace of the present disclosure apart is how the knee brace (of the present disclosure) adjusts its length in response to knee movement. When the knee extends, the brace elongates; and when the knee bends, the brace shortens. This change in length reduces the load placed on the knee by creating a separative force between the upper and lower leg. Unlike traditional braces that act like a cast to immobilize the joint, this brace operates more like crutches, actively supporting the knee while allowing movement.

A novel feature of the knee brace of the disclosure is in the brace's offset hinge. The offset hinge can be positioned either in front of or behind the knee's natural pivot point. As the knee bends or straightens, the brace extends and shrinks at a different rate than the knee, while still mirroring the rotation. When the leg straightens, the brace grows longer, reducing the pressure on the knee joint. Conversely, when the knee contracts, the brace becomes shorter, decreasing its tightness around the leg and allowing more flexibility. This dynamic response not only reduces stress on the knee, but also makes the brace more comfortable when less support is needed. This dynamic change in length also allows the brace to “hone” in closer to the knee with each step, allowing it to recenter.

By anchoring to the muscles above and below the knee, the brace creates a separative force that mimics the knee's natural soft tissue function. Because the hinge of the brace is positioned off-center to the hinge of the knee, the hinge of the brace also engages before the knee, reducing the impact when landing on your foot.

Described below are the brace's various components and details regarding assembly of the various components.

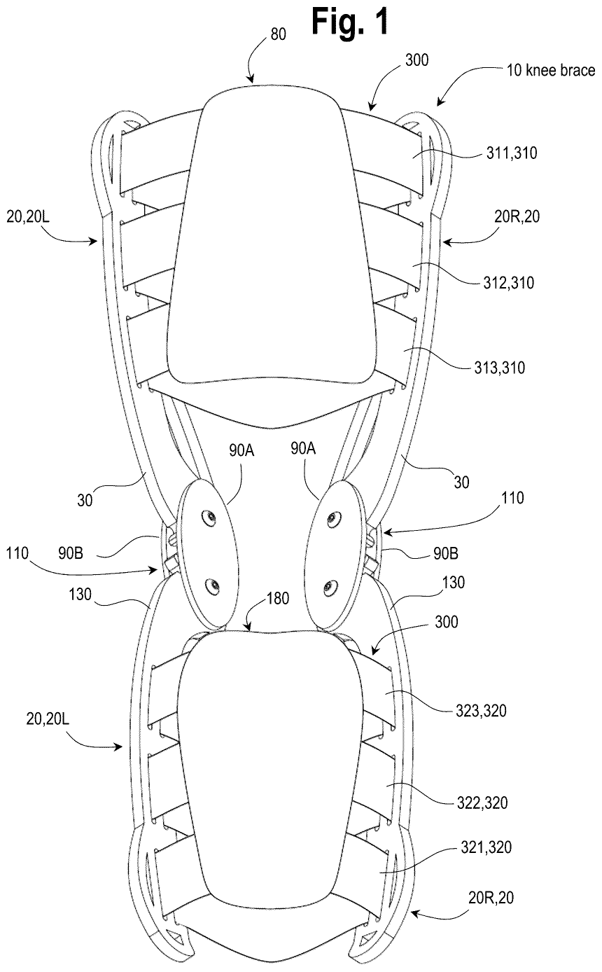

FIG. 1 is a back perspective view of a knee brace 10, in accordance with principles of the disclosure. As described in detail below, the knee brace 10 can include side assemblies 20. The side assemblies 20 can include a left side assembly 20L and a right side assembly 20R, which oppose each other. Each of the side assemblies 20 can be of similar or same construct, and/or mirror image construct. Each of the side assemblies 20 can include an upper cheek structure or upper cheek 30 and a lower cheek structure or lower cheek 130.

Each of the side assemblies 20, as described below, can further a respective offset hinge or hinge assembly 110. The offset hinge assembly 110 can provide a pivot point that can connect the respective upper cheek 30 to the lower cheek 130, in each of the opposing side assemblies 20.

The knee brace 10 can include straps that wrap around the user's leg, and padding that cushions the straps. As noted above, the knee brace 10 may include the same or similar construction on opposing sides and/or mirrored construct on the left side and the right side of the knee brace.

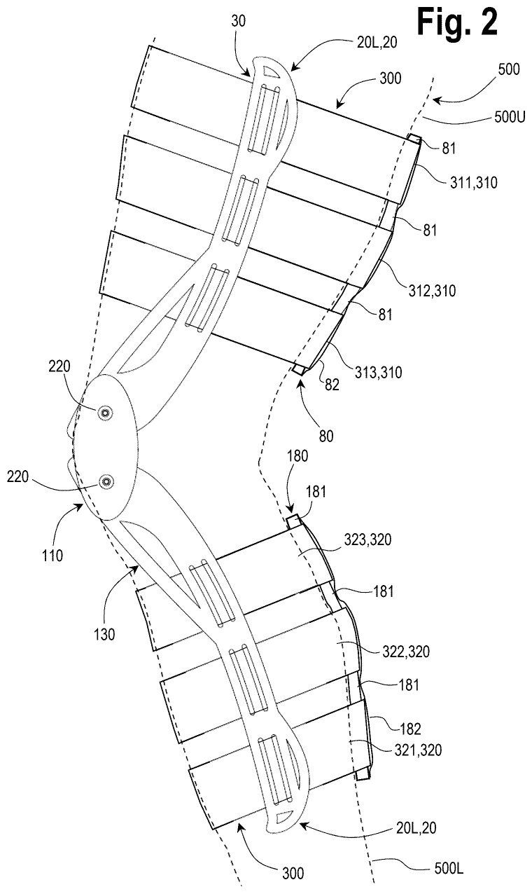

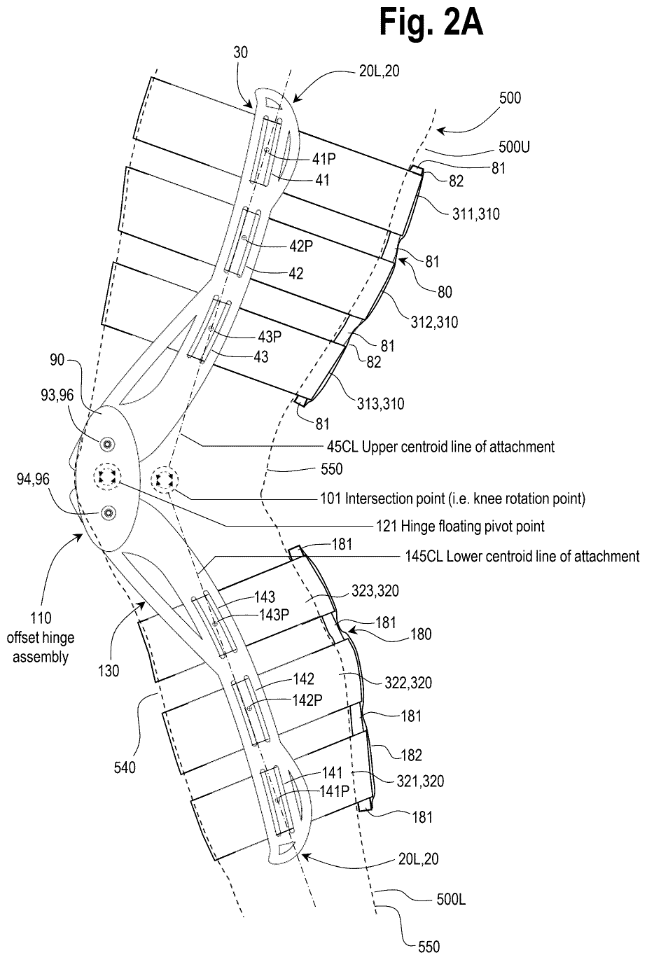

The brace may use a series of upper straps 310 and a series of lower straps 320, that thread through the side assemblies 20 on both sides-so as to attach the knee brace 10 to the leg. An upper padding assembly 80 and a lower padding assembly 180 can provide comfort and adjust to the leg's contours. One or more of the padding assemblies 80, 180 may be removed, however, in some embodiments-so as to provide further range of motion when the leg folds. One or more of the padding assemblies 80, 180 may be removed, in a situation that the user's clothing might provide any needed cushioning or padding. As shown in FIG. 2, the padding 80 can include a base layer 81 (of cushion or padding material) and a top layer 82, with the top layer being cloth, for example. Accordingly, the straps 310 can be positioned or sandwiched between the base layer 81 and the top layer 82. The base layer 81 and the top layer 82 can be attached via adhesive and/or stitching, or can be integrally formed, for example.

Relatedly, as shown in FIG. 2, the padding 180 can include a base layer 181 (of cushion or padding material) and a top layer 182, with the top layer being cloth, for example. Accordingly, the straps 320 can be positioned or sandwiched between the base layer 181 and the top layer 182.

The base layer 181 and the top layer 182 can be attached via adhesive and/or stitching, or can be integrally formed, for example.

As noted above, FIG. 1 is a back perspective view of a knee brace 10. The offset hinge 110, for each of the side assemblies, can be positioned in front of or behind the knee's natural pivot point, allowing the knee brace 10 to extend when the knee is straight and contract when the knee bends.

The offset hinge 110 is shown, in the illustrative embodiment of FIG. 1, to extend beyond the knee in front, i.e. in front of the knee.

Each of the left and right side assemblies 20 can be secured to the leg using the upper strap assemblies 310 and lower strap assemblies 320. The strap assemblies 300 can be threaded or weaved through the side assemblies 20 (as shown in FIG. 2, for example, and described below) to ensure a secure fit of the knee brace 10 onto the leg of the user. The tightness of the straps can be adjusted depending on the wearer's need for either rigidity or flexibility. In some cases, the brace can be worn with a leg compression sleeve (not shown) for additional stability and/or comfort.

An advantage of the knee brace of the disclosure is that the knee brace 10 can relieve pressure on the knee during walking and other activities. The knee brace 10 can be adjusted to accommodate different leg shapes and sizes. The padding and straps, with regard to shape and/or thickness for example, can be customized to provide both comfort and stability, allowing the knee brace 10 to be worn for extended periods without causing discomfort or restricting movement.

FIG. 2 is side view of a knee brace of the disclosure, the same or similar to the knee brace of FIG. 1, mounter on a leg of a user. FIG. 2 shows, in accordance with at least one embodiment of the disclosed subject matter, a possible orientation of the knee brace 10 on the leg of a user.

FIG. 2A is schematic side view a knee brace of the disclosure, the same or similar to the knee brace of FIG. 2, mounted on a leg of a user. FIG. 2A shows a brace rotation point which can be described as a hinge floating pivot point 121. FIG. 2A also shows a knee rotation point 101, as described further below. FIG. 2A also shows the relative positioning between the hinge floating pivot point 121 and the knee rotation point 101. By shifting the hinge floating pivot point 121 either in front of or behind the knee rotation point 101, the knee brace 10 can create a significant difference, i.e. a differential, in the length, i.e. a “lever-arm length” of the knee brace 10 compared to the leg itself. This differential allows the brace to reduce the load on the knee by absorbing much of the stress that would normally be transferred to the joint-redistributing forces along the length of the leg, particularly during activities that require repetitive motion or high impact.

The offset of the hinge floating pivot point 121 can be between 0.5 inches and 10 inches, for example, away from the knee's natural pivot point, or the center of the knee, i.e. the knee rotation point 101. The offset of the hinge floating pivot point 121 relative to the knee rotation point 101 can be 0.5-1.5 inches; 1.5-2.5 inches; 2.5-3.5 inches; 3.5-4.5 inches; 4.5-5.5 inches; 5.5-6.5 inches; 6.5-7.5 inches; 7.5-8.5 inches; 8.5-10 inches, for example, in accordance with at least some embodiments of the disclosure. Such offset can be based on physical attributes of the intended user, amount of separative force desired, and/or other attributes.

As shown in FIG. 2A, the front of the leg 540, back of the leg 550, upper leg 500U, and lower leg 500L are shown in relation, i.e. in reference, to the brace 10, showing how the brace 10 can align with the natural contours of the leg. Further details are described below.

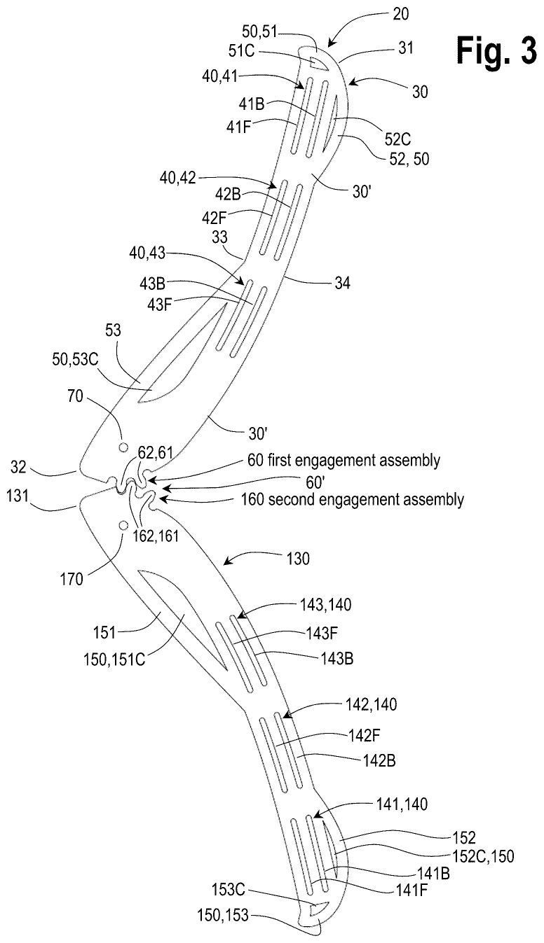

FIG. 3 is a side view of a side assembly 20 the same as or similar to a side assembly of the knee brace of FIG. 1, in accordance with at least one embodiment. FIG. 3 shows further details, as described below, to the various components that can make up the upper cheek 30 and the lower cheek 130. FIG. 3 shows a gear connection 60′ that allows the upper and lower cheeks 30, 130 to interface and “push off” each other. The gear connection 60′ can ensure the controlled movement, including rotational movement, of the brace during flexion and extension. The gear connection 60′ can also contribute to the operation of the offset hinge 110, which drives the unique motion of the knee brace. The gear connection 60′ can include a first gear rack 61 of the upper cheek (on both sides of the knee brace 10), as well as a second, mating second gear rack 161 of the lower cheek 130, on both sides of the knee brace 10.

The upper cheek slots 40 and lower cheek slots 140 provide a way, i.e. provide structure, to thread or weave respective straps through a set of slots, so as to secure the brace to the leg, as shown in FIG. 1, for example. The upper straps 310 connecting to the upper leg 500U feed through the upper cheek slots 40. The lower straps 320 connecting to the lower leg 500L feed through the lower cheek slots 140.

As shown in FIG. 3, various struts 50, 150, further described below, can provide reinforcement and structural strength throughout the structure, ensuring that stress and strain from the gear connection 60′ are evenly distributed throughout the knee brace, and to the leg of the user. FIG. 3 also shows shaft holes or pin holes 70, 170 in both the upper cheek 30 and the lower cheek 130.

The shaft holes 70, 170 provide for attachment of the upper cheek 30 to the lower cheek 130. In particular, the shaft holes 70, 170 provide for the upper cheek 30 and the lower cheek 130 to be connected. The upper cheek 30 and the lower cheek 130, of both sides, can be connected by a respective connector assembly 89, so as to provide smooth and controller motion between the upper cheek 30 and the lower cheek 130, and to mechanically tie the upper cheek 30 and the lower cheek 130. The various components can work together to form the offset hinge 110, and so as provide the knee brace's ability to separate the upper and lower leg of the user during movement.

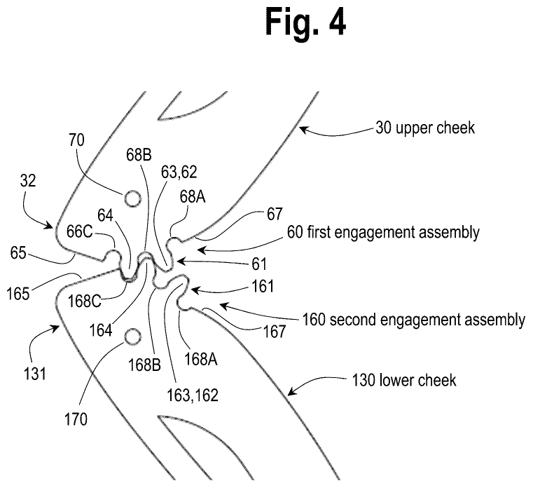

FIG. 4 is a partial side view of the side assembly 20 of the knee brace of FIG. 3. Further details are described below.

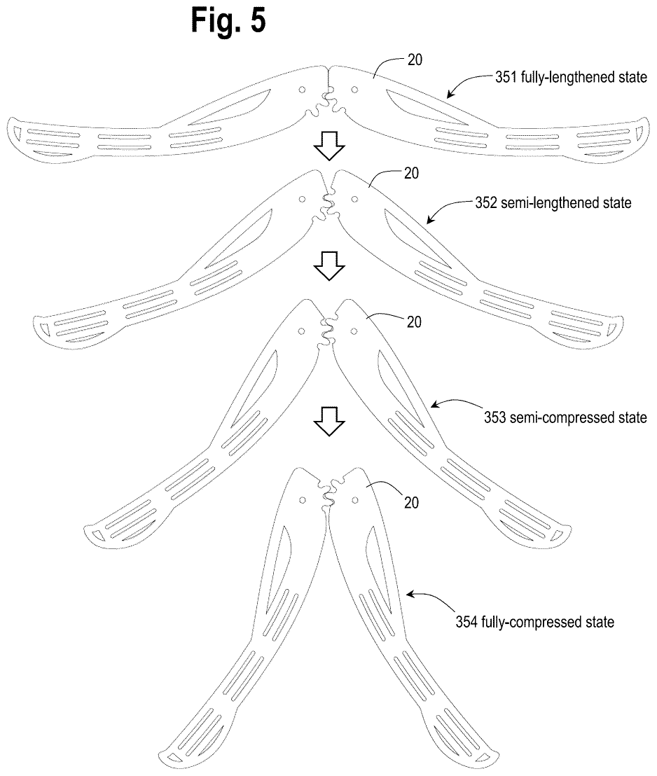

FIG. 5 is a flow schematic state diagram showing different states, i.e. different operational dispositions of a side assembly 20 of the knee brace 10. That is, FIG. 5 shows various states of knee brace 10 between extension and contraction, from a fully lengthened state 351; to semi-lengthened state 352; to semi-compressed state 353; to fully-compressed state 354.

The transition or movement between these states is controlled by the offset hinge 110, and the extension of the leg. As the brace moves from a compressed state to a more lengthened state, the offset hinge 110 produces a separative force, i.e. a separative force between the upper leg and lower leg, in accordance with one novel aspect of the disclosure. As a result of mechanics of the offset hinge 110 and the structure of the offset hinge 110, during rotation of the knee brace 10 from a first state to a more lengthened state, the knee brace 10 lengthens at a greater rate than does the human knee. Accordingly and relatedly, the construct of the offset hinge 110, i.e. the offset hinge assembly, provides for the knee brace 10 to become fully extended before the knee becomes fully extended. As a result, reduction of the load on the knee before and leading up to full extension of the knee occurs. Such reduction of the load on the knee can assist a user in performing various physical activities including moving between a variety of stances, positions, and orientations, walking and running, for example, while reducing stress and pressure on the user's knee. As a result, the user may be able to perform activities more comfortably with the knee brace 10, than if the knee brace 10 were not used.

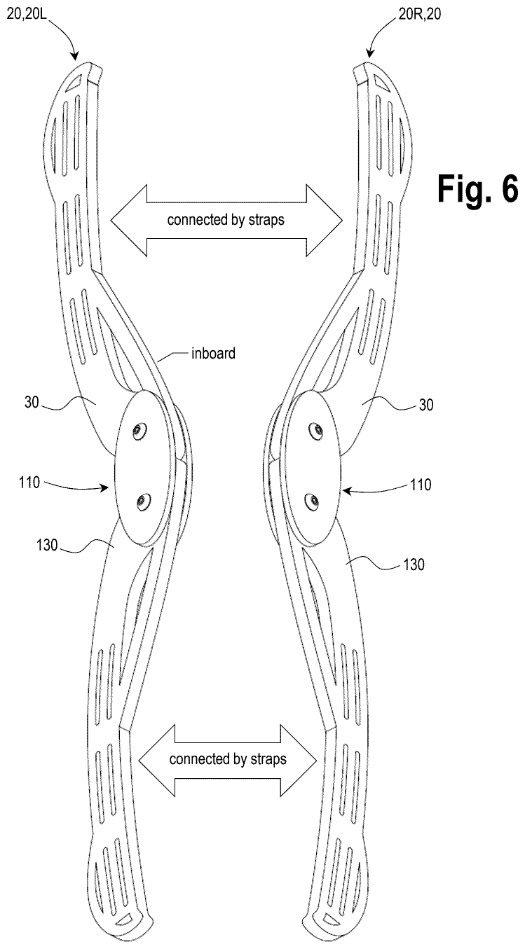

FIG. 6 is a schematic diagram of opposing side assemblies 20, the same as or similar to the side assemblies 20 of FIG. 1, without straps, in accordance with principles of the disclosure. More specifically, FIG. 6 shows opposing side assemblies 20 including a left side assembly 20L and a right side assembly 20R. Each of the side assemblies 20 include a respective offset hinge 110.

The left side assembly 20L and the right side assembly 20R can be the same construct and/or can be mirror image from each other. Each of the side assemblies 20 can be constructed of a flat, rigid material, in accordance with at least one embodiment of the disclosure. Each of the side assemblies 20 can be connected by an offset hinge 110. Each offset hinge 110 can include a pair of plates or connector plates 90. That is, opposing ends of the upper cheek 30 and the lower cheek 130 can be connected by a pair of plates. More specifically, a lower end of the upper cheek 30 and an upper end of the lower cheek 130 can be “sandwiched” between the plates 90.

The plates 90 can be attached together using pin assemblies 220 or pins 220.

The upper cheek 30, the lower cheek 130 and the plates 90 can all be in the form of respective flat plates that are laid on top of one another. However, the disclosure is not limited to such particulars. That is, the cheeks 30, 130 and the plates 90 are not limited to respective flat plates.

Other shapes and or geometries may be utilized, so as to be more aesthetically pleasing and/or to be more custom fit to a particular user's leg, for example. As described further below, the cheeks 30, 130 and the plates 90—along with the pair of pin assemblies 220—form the offset hinge or offset hinge assembly 110.

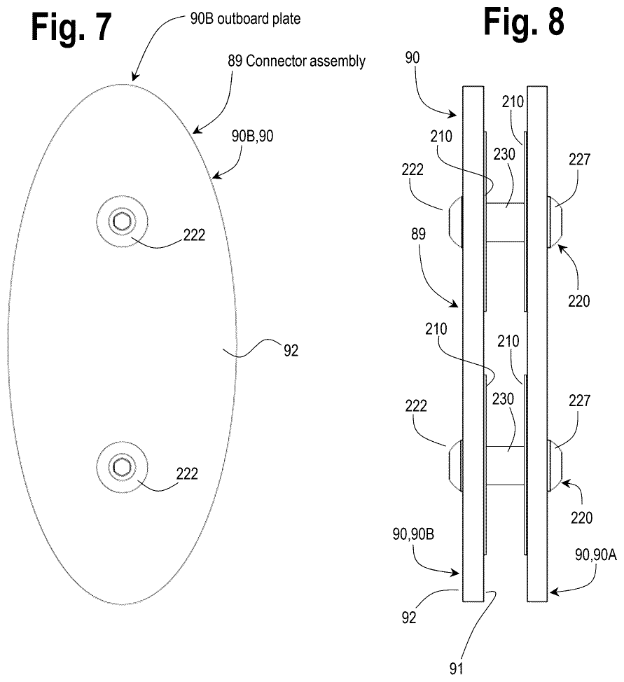

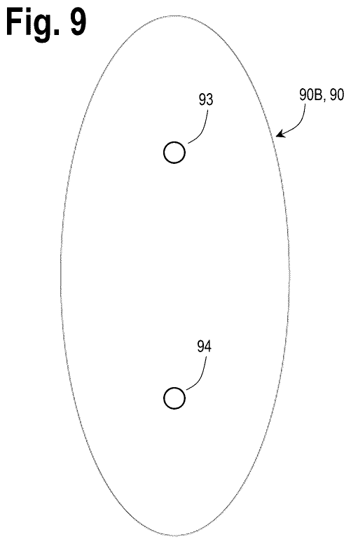

FIG. 7 is a side view of a connector assembly 89. Relatedly, FIG. 8 is a front view of a connector assembly 89, in accordance with at least one embodiment. FIG. 9 is a side view of a plate 90, without pins 220, of FIG. 7. The connector assembly 89 connects or links the lower end of the upper cheek 30 to the upper end of the lower cheek 130, so that the cheeks 30, 130 are hingedly connected. Further details are described below.

FIG. 8 provides a detailed view of a rigid body connection, i.e. the connection assembly 89, that links the rigid upper body (i.e. cheek 30) and rigid lower body (i.e. cheek 130). This connection can include an outboard plate 90B and an inboard plate 90A that are positioned on opposing sides of the upper and lower rigid bodies, i.e. components 30, 130. Washers 210 can be placed between these plates to allow for smooth movement, while horizontal bolts, pins, or pin assemblies 220 secure the plates to the cheeks 30, 130 to ensure the assembly remains intact.

This design can ensure the rigid body connection, i.e. the connection assembly 89, accommodates the flexibility required for knee movement without compromising the strength and stability of the brace. The rigid body connection 89, or parts thereof, may be an integral piece in the function of the offset hinge, allowing the upper and lower rigid bodies to move relative to one another as the knee bends and extends. The construct of the connection assembly 89, including the construct of the plates 90 and pin assemblies 220, provides the ability to stack the upper cheek 30 and the lower cheek 130 in line with each other, thereby improving strength of the knee brace 10.

The combination of the knee brace 10 including the offset hinge 110 provides a structure and solution that can reduce pressure and strain on a user's knee and that can provide the ability of a user to do certain physical activities that the user would not otherwise be able to do, i.e. without the knee brace 10. By creating a separative force between the upper and lower leg, the brace 10 can reduce the load on the knee while allowing for enhanced mobility. In particular, the construct and design of the knee brace 10 allows for the brace to extend, when straightened, at a greater rate than the knee, thus cushioning the knee joint during movement. The construct and design of the various components of the knee brace 10 helps to ensure that the brace 10 remains comfortable, stable, and effective.

Hereinafter, further details of the knee brace 10 will be described with reference to FIGS. 1-4, in particular. As described above, the knee brace 10 can include a pair of opposing side assemblies 20. The side assemblies 20 can include a left side assembly 20L and a right side assembly 20R.

The left side assembly 20L and a right side assembly 20R can be of the same or similar construct and can be mirror image of each other. The side assemblies 20 can be connected with straps 300.

Each side assembly 20 can include an upper cheek 30. The upper cheek 30 can also be described as a rigid body, leg cheek, leg plate, or plate. The upper cheek 30 can include a body 30′. The body 30′ can be curved to the front, so as to offset the brace rotation point 121 from the knee rotation point as described herein, as is shown in FIG. 2A, for example. The body 30′ can include an upper end 31 and a lower end 32. The body 30′ can also include a back edge 33 and a front edge 34.

As shown in FIG. 3, for example, the upper cheek 30 can include upper cheek slots, upper slots, or slots 40, which connect to upper straps 310.
The upper cheek slots 40 can include first slots 41 (i.e. outboard slots 41) that include front slot 41F and back slot 41B. The first slots 41 can provide attachment to a first strap 311.
The upper cheek slots 40 can include second slots 42 (i.e. middle slots 42) that include front slot 42F and back slot 42B. The second slots 42 can provide attachment to a second strap 312.
The upper cheek slots 40 can include third slots 43 (i.e. inboard slots 43) that include front slot 43F and back slot 43B. The third slots 43 can provide attachment to a third strap 313.

Accordingly, the upper check slots can include the first slots 41, the second slots 42 and the third slots 43. The upper straps 310 can include the first strap 311, the second strap 312 and the third strap 313.

With further reference to FIG. 3, the lower cheek 130 can also include cheek slots. That is, the lower cheek 130 can include lower cheek slots, lower slots, or slots 140, which connect to lower straps 320.
The lower cheek slots 140 can include first slots 141 (i.e. outboard slots 141) that include front slot 141F and back slot 141B. The first slots 141 can provide attachment to a first strap 321 of the lower straps 320.
The lower cheek slots 140 can include second slots 142 (i.e. middle slots 142) that include front slot 142F and back slot 142B. The second slots 142 can provide attachment to a second strap 322 of the lower straps 320.
The lower cheek slots 140 can include third slots 143 (i.e. inboard slots 143) that include front slot 143F and back slot 143B. The third slots 143 can provide attachment to a third strap 323 of the lower straps 320.

Accordingly, the lower check slots can include the first slots 141, the second slots 142 and the third slots 143. The lower straps 310 can include the first strap 321, the second strap 322 and the third strap 323.

Accordingly, the upper straps 310, for example, can provide attachment of each upper cheek 30, on opposing sides, to the user's leg above the knee. As illustrated in FIG. 2A, the upper strap slots 40 can be positioned about and serve to define an “upper centroid line of attachment” 45CL of each of the upper cheeks 30 to a user's leg. In other words, the upper cheek slots 40 are positioned about and define the centroid line of attachment 45CL such that the upper cheeks 30 tend, in a physical sense in this embodiment, to be positioned about and aligned with the user's leg along the centroid line of attachment 45CL. Given such alignment with the user's leg, the centroid line of attachment 45CL tends to be pass through or pass near the knee rotation point of the human user.

To explain further, in accordance with one embodiment, an upper center point 41P can be defined as a center point between the slots 41F, 41B of the first slots 41. A lower center point 43P can be defined as a center point between the slots 43F, 43B of slots 43. The centroid line of attachment 45CL can be defined as a line passing through the upper center point 41P and the lower center point 43P.

The centroid line of attachment 45CL can also be defined in terms of the upper center point 41P and the lower center point 43P, as well as a middle center point 42P. That is, a middle center point 42P can be defined as a center point between the slots 42F, 42B of the second slots 42.

Accordingly, the centroid line of attachment 45CL can be defined as a straight line that runs through the “middle” or centroid, of the points 41P, 42P and 43P, such that the centroid line of attachment 45CL represents an average path about the points 41P, 42P and 43P. In other words, under this interpretation, the centroid line of attachment 45CL can be calculated based on the position of the points 41P, 42P and 43P.

As described above, the upper slots 40 can be positioned about the centroid line of attachment 45CL such that the upper cheek 30 tends, in a physical sense in this embodiment, to be positioned about and aligned with the user's leg along the centroid line of attachment 45CL.

Given such alignment with the user's leg, the centroid line of attachment 45CL tends to pass through or pass near the knee rotation point of the human user. It is appreciated that the particular physical geometry and dimensions of the user will dictate the particular alignment of the upper cheeks 30, i.e. the cheeks 30 on opposing sides, with the user's leg. However, as described herein, the centroid line of attachment 45CL can be defined relative to the strap slots 40, in accordance with at least one embodiment. The centroid line of attachment 45CL can be defined relative to upper slots 41 and the lower slots 43; relative to upper, lower, and middle slots (of the slots 40); or relative to some other number of strap slots in the upper cheek 30, if there are more than three sets of slots in the upper cheek 30.

In similar manner to the slots 40 of the upper cheek 30, the slots of the lower cheek 130 can also define a centroid line of attachment 145CL. A lower center point 141P can be defined as a center point between the slots 141F, 141B of the first slots 141. An upper center point 143P can be defined as a center point between the slots 143F, 143B of slots 143. The centroid line of attachment 145CL can be defined as a line passing through the lower center point 141P and the upper center point 143P, of the lower cheek 130. The centroid line of attachment 145CL, of the lower cheek, can be determined in manner as described with reference to the centroid line of attachment 45CL, of the upper cheek.

The centroid line of attachment 45CL (of the upper cheek 30) and the centroid line of attachment 145CL (of the lower cheek 130) will intersect at an intersection point 101, as illustrated in FIG. 2A.

It is appreciated that such intersection point 101 may or may not exactly coincide with the actual physical knee rotation point of the human user. However, the physicality of the knee brace 10 is based on an understanding that the intersection point 101 is positioned at or near the knee rotation point 530. However, of note, the intersection point 101 is clearly positioned behind (in the embodiment of FIG. 3 for example) a pivot point of the offset hinge 110.

In accordance with one embodiment of the disclosure, the hinge rotation point 121 can be defined as a point between the upper pin hole 93 and a lower pin 94 of a connector plate 90.

Such connector plate 90 and the manner in which the connector plate 90 connects the upper cheek 30 with the lower cheek 130 is described below. The hinge rotation point 121 can be described as a “hinge floating pivot point” in that such pivot point is not a set point, but rather “floats” as the upper cheek 30 is rotated relative to the lower cheek 130.

It is appreciated that the above disclosure relating to the upper centroid line of attachment 45CL and the lower centroid line of attachment 145CL is based on an understanding that, with the embodiment of FIG. 2A, the cheeks 30, 130 are aligned with the user's leg. However, in accordance with at least some embodiments of the disclosure, the cheeks 30, 130 need not be so aligned with the user's leg, i.e. the upper cheeks 30 may constructed so as to not be aligned with the user's upper leg and the lower cheeks 130 may constructed so as to not be aligned with the user's lower leg. In such a construct, the positioning of the cheeks 30, 130 would not be dispositive of the center or centroid of the upper leg of the user and the lower leg of the user, respectively. Rather, a centroid of the straps configured to be wrapped around the user's leg can be assessed to be indicative of the center or centroid of the upper leg of the user and the lower leg of the user.

To explain further, with the knee brace of claim 1, the first upper strap (e.g. strap 311 as shown in FIG. 1) can be connected to the left upper cheek and the right upper cheek, the first upper strap configured to encircle the upper leg of the user so as to have a first centroid defined by first material of the first upper strap, as such first material is positioned about the upper leg.

The second upper strap (e.g. strap 313 as shown in FIG. 1) can be connected to the left upper cheek and the right upper cheek, the second upper strap configured to encircle the upper leg of the user so as to have a second centroid defined by second material of the second upper strap, as such second material is positioned about the upper leg. The first centroid and the second centroid can define an upper centroid line of attachment.

The third lower strap (e.g. strap 323 as shown in FIG. 1) can be connected to the left lower cheek and the right lower cheek, the third lower strap configured to encircle the lower leg of the user so as to have a third centroid defined by third material of the third lower strap, as such third material is positioned about the lower leg.

The fourth lower strap (e.g. strap 321 as shown in FIG. 1) can be connected to the left lower cheek and the right lower cheek, the fourth lower strap can be configured to encircle the lower leg of the user so as to have a fourth centroid defined by fourth material of the fourth lower strap, as such fourth material is positioned about the lower leg. The third centroid and the fourth centroid can define a lower centroid line of attachment.

Relatedly, the upper centroid line of attachment and the lower centroid line of attachment can be said to be rotatable about a second hinge axis, and the second hinge axis being offset from the first hinge axis.

If additional straps are employed, a centroid relative to such additional straps can be determined and used in determination of the upper centroid line of attachment and/or the lower centroid line of attachment, depending on whether the strap is an upper strap or a lower strap.

The “centroid” of a strap can be defined as a center point of the strap in space about which the strap is positioned when the strap is configured, in conjunction with other components of the knee brace, to be wrapped around the leg of a user, in accordance with at least one embodiment of the disclosure. It is appreciated that, in at least some embodiments of the disclosure, the three straps 311, 312, 313 (i.e. the three upper straps) shown in FIG. 2A, for example, could be instead a single strap. It is appreciated that, in at least some embodiments of the disclosure, the three straps 321, 322, 323 (i.e. the three lower straps) shown in FIG. 2A, for example, could be instead a single strap.

In general, it is appreciated that the number of straps can be varied as desired. As noted above, a knee brace of the disclosure can include one upper strap to connect the upper cheeks 30, and one lower strap to connect the lower cheeks 130. For example, a knee brace of the disclosure can include two upper straps to connect the upper cheeks 30, and two lower straps to connect the lower cheeks 130. For example, a knee brace of the disclosure can include three upper straps to connect the upper cheeks 30, and three lower straps to connect the lower cheeks 130, as is shown in FIGS. 1, 2 and 2A.

For each strap, a suitable slot can be provided in a corresponding upper cheek or lower cheek, so as to secure the particular strap to the corresponding upper cheek 30 or lower cheek 130.

As described herein, the upper cheek 30 and the lower cheek 130 (of each of the left side assembly 20L and the right side assembly 20R) can be connected, i.e. mechanically connected or attached, to each other by a connector assembly 89, as shown in FIG. 1 and FIG. 2, for example.

The connector assembly 89 can include connector plates 90. Further details of the connector assembly 89, including the connector plates 90, are described below with reference to FIGS. 7-9.

As described above, FIG. 7 is a side view of a connector assembly 89. FIG. 9 is a side view of a plate 90, with connector assemblies 222 removed. Further, FIG. 8 is a front view of the connector assembly 89.

As shown in FIGS. 7 and 8, each connector assembly 89 can include a pair of connector plates 90.

Each connector plate 90 can also be described as a plate or bridge plate 90. Relatedly, the upper cheek 30 can include a shaft hole 70. The shaft hole 70 can be positioned at a lower end 32 of the upper cheek 30, as shown in FIG. 3. The lower cheek 130 can include a shaft hole 170. The shaft hole 70 can be positioned at an upper end 131 of the lower cheek 130. As described below, the shaft holes 70, 170 can serve to connect to the connector assembly 89.

With further reference to FIGS. 7 and 8, the set of connector plates 90 can include an inboard plate 90A and an outboard plate 90B. The connector plates 90A, 90B can be positioned on opposing sides of the cheeks 30, 130—so that the cheeks 30, 130 are positioned between or “sandwiched” between the connector plates 90A, 90B. Such arrangement is shown in FIG. 1 and FIG. 2, for example.

As shown in FIG. 8, each connector plate 90 can include an inboard side 91 and an outboard side 92. Such nomenclature relates to the interrelationship of each connector plate 90 relative to the cheeks 30, 130 that are connected by the plates 90. Accordingly, the inboard side 91 of each connector plate 90 is positioned against or facing the cheeks 30, 130. On the other hand, the outboard side 92 of each connector plate is “outboard” or facing outward relative to the cheeks 30, 130. A washer 210 can be positioned between each plate 90 and the cheeks 30, 130, as is shown in FIG. 8.

Each connector plate 90 can include an upper pin hole, shaft hole, or hole 93, as shown in FIG. 9.

The upper pin hole 93 can receive an attachment pin or pin assembly 220. The pin assembly 220 can also pass through the pin hole 70 at the lower end of the upper cheek 30. Accordingly, the pin assembly 220 can pass through the upper pin hole 93 of the outboard plate 90B; the pin hole 70 of the upper cheek 30; and the upper pin hole 93 of the inboard plate 90A, so as to connect such components together. Accordingly, the lower end of the upper cheek 30 can be sandwiched between the outboard plate 90B and the inboard plate 90A, so as to be rotatably attached to the connector assembly 89. Washers 210 can be positioned between the plates 90 and the upper cheek 30 so as to provide smooth rotation of the plates 90 against the cheek 30.

Each connector plate 90 can include a lower pin hole, shaft hole, or hole 94, as shown in FIG. 9.

The pin hole 94 can receive an attachment pin or pin assembly 220, i.e. a second pin assembly 220. The pin assembly 220 can pass through the pin hole 170 at the upper end of the lower cheek 130. Accordingly, a pin assembly 220 can pass through the lower pin hole 94 of the outboard plate 90B; the pin hole 170 of the lower cheek 130; and the lower pin hole 94 of the inboard plate 90A, so as to connect such components together. Accordingly, the upper end of the lower cheek 130 can be sandwiched between the outboard plate 90B and the inboard plate 90A, so as to be rotatably attached to the connector assembly 89. Washers 210 can be positioned between the plates 90 and the lower cheek 130 so as to provide smooth rotation of the plates 90 against the lower cheek 30. FIG. 11 is a side view of a washer 210 the same as or similar to the washer of FIG. 8, in accordance with principles of the disclosure. The washer 210 can include a hole or bore 211, and an outer diameter (OD) 212, as shown in FIG. 11.

Accordingly, the upper cheek 30 can be rotatably connected to the lower cheek 130 via the connector assembly 89. Rotation of the upper cheek 30 relative to the lower cheek 130 can be controlled or modulated by an engagement assembly (or engagement structure) 60 of the upper cheek 30 and an engagement assembly (or engagement structure) 160 of the lower cheek 30.

As shown in FIG. 4, for example, the upper cheek 30 can include an engagement assembly 60.

The engagement assembly 60 can also be described as a first engagement assembly, an engagement structure or engagement portion, for example. The engagement assembly 60 can serve to rotatably engage the lower end 32 (of the upper cheek 30) with an engagement assembly 160 at the upper end 131 of the lower cheek 130.

The engagement assembly 60 can include a gear rack 61 as shown in FIG. 4. The gear rack 61 can be positioned at the lower end 32 or constitute a part of the lower end 32 of the body 30′ of the upper cheek 30. The gear rack 61 can include gears 62. The gears 62 can include a back gear 63 and a front gear 64. In some embodiments, additional gears can be included. The engagement assembly 60 can also include grooves or recesses between the gears 62. Such grooves can include back addendum or groove 68A, center groove or addendum 68B, and front groove or addendum 66C.

The gears 62 can, in operation of the knee brace 10, engage with mating gears 162 of the lower cheek 130.

The lower cheek 130 can include an engagement assembly 160. The engagement assembly 160 can also be described as a second engagement assembly, an engagement structure or engagement portion, for example. The engagement assembly 160 can serve to rotatably engage the upper end 131 (of the lower cheek 130) with the engagement assembly 60 at the lower end 32 of the upper cheek 130.

The engagement assembly 160, i.e. the second engagement assembly 160, can include a gear rack 161 as shown in FIG. 4. The gear rack 161 can be positioned at the upper end 131 or constitute a part of the upper end 131 of the body 130′ of the lower cheek 130. The gear rack 161 can include gears 162. The gears 162 can include a back gear 163 and a front gear 164. In some embodiments, additional gears can be included. The engagement assembly 160 can also include grooves or recesses between the gears 162. Such grooves can include back addendum or groove 168A, center groove or addendum 168B, and front groove or addendum 166C.

The gears 162 can, in operation of the knee brace 10, engage with mating gears 62 of the upper cheek 30.

With further reference to FIG. 4, the engagement assembly 60, of the upper cheek 30, can also include a front stop surface 65. The front stop surface 65 can engage with a mating front stop surface 165 of the lower cheek 130. The stop surfaces 65, 165, as shown in FIG. 4, can serve to limit or restrict rotation of the upper cheek 30 relative to the lower cheek 130. The stop surfaces 65, 165 can serve to limit extension or elongation of the upper cheek 30 relative to the lower cheek 130 past a desired rotational limit. Such construct can constrain rotation to provide, but not extend past, the fully lengthened operational state as shown at 351, in FIG. 5, for example.

The engagement assembly 60 can also include a curved back surface 67. The curved back surface 67 can serve to provide clearance so that the upper cheek 30 and the lower cheek 130 can be rotated to a fully compressed operational state 354, as shown in FIG. 5.

In similar manner, the engagement assembly 160 can also include a curved back surface 167.

The curved back surface 167 can serve to provide clearance so that the upper cheek 30 and the lower cheek 130 can be rotated to a fully compressed operational state 354, as shown in FIG. 5.

Accordingly, the engagement assembly 60 (of the upper cheek 30) can engage with the engagement assembly 160 (of the lower cheek 130). Such engagement of the engagement assembly's 80, 180 can serve to control rotational disposition of the upper cheek 30 vis-à-vis the lower cheek 130.

As described herein, a pin or pin assembly 220 can be used to connect the plates 90 to the cheeks 30, 130. The pin or pin assembly 220, i.e. an attachment pin, can be in various forms. The pin or pin assembly 220 can be in the form of a pin, bolt, shaft, threaded shaft, pin with hole for Cotter key, Cotter key pin, or any other pin or similar construct that will serve to connect the connector plate 90 to the cheeks 30, 130. For example, if the attachment pin 96 is in the form of a bolt, the bolt may include a head and threaded shaft upon which a nut can be screwed. The nut may be a locking, nylon nut, for example. It is appreciated that whatever pin or pin assembly 220 is utilized, the structure may be low profile so as to not interfere with operation of the knee brace and so as to not “catch” on clothes that the user is wearing or other objects.

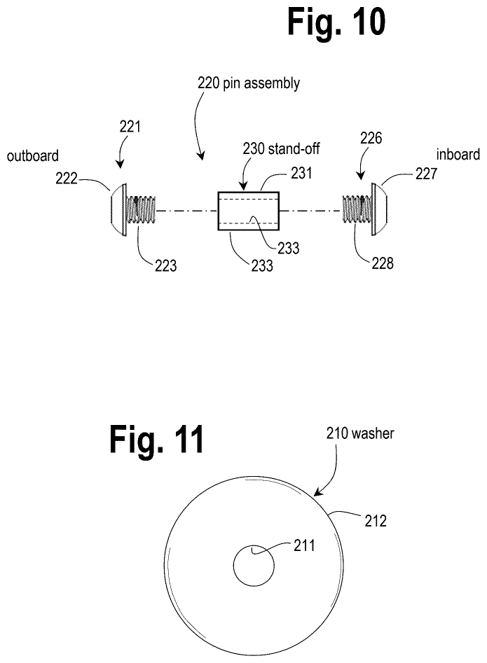

As described above, FIG. 8 shows the pin or pin assembly 220, as described herein. FIG. 10 is an exploded view of a pin or pin assembly 220, in accordance with at least one embodiment. The pin assembly 220 can include a stand-off 230 with a cylindrical body 231. The cylindrical body 231 can be include an internally threaded bore 233. The stand-off 230 can include an outer surface 232 that is cylindrical. A first screw or machine screw 221 (with head 222 and threaded shaft 223) can be threadably received into a first end of the stand-off 230. A second screw or machine screw 226 (with head 227 and threaded shaft 228) can be threadably received into a second end of the stand-off 230. The first screw 221 and the second screw 226 can be identical in structure. For purposes of illustration, the first screw 221 can be an outboard attachment screw (facing outboard of the knee brace 10), and the second screw 26 can be an inboard attachment screw.

As shown in FIG. 3, for example, the upper cheek 30 and the lower cheek 130 can be provided with various reinforcing structure or be constructed in a manner so as to enhance the structural strength and rigidity of the cheeks 30, 130. Such a reinforcing structure might include a rib, strut, truss, thickened edge portion, or other reinforcing structure that may be desired. The upper check 30 might be constructed of polycarbonate plastic.

In the embodiment of FIG. 3, the body 30′ can include a plurality of struts 50. The struts 50 can include a top strut 51, as shown in FIG. 5. The top strut 51 can be curved and provided at a top of the upper end 31 of the body 30′. The top strut 51 can include or be defined by an opening, cavity or web opening 51C.

The struts 50 can include a back strut 52, as shown in FIG. 3. The back strut 52 can be curved and provided at a top, back of the upper end 31 of the body 30′. The back strut 52 can include or be defined by an opening, cavity or web opening 52C.

The struts 50 can include a front strut 53, as shown in FIG. 5. The front strut 53 can be curved or straight and provided at or constitute a part of the front edge 34 of the body 30′ of the upper cheek 30. The front strut 53 can include or be defined by an opening, cavity or web opening 53C.

In similar manner to the upper cheek 30, the lower cheek 130 can also include struts, i.e. the lower cheek 130 can include struts 150.

The struts 150 can include a top strut 151, as shown in FIG. 3. The top strut 151 can be curved and provided at a top, front of the upper end 131 of the body 130′. The top strut 161 can include or be defined by an opening, cavity or web opening 151C.

The struts 150 can include a back strut 152, as shown in FIG. 3. The back strut 152 can be curved and provided at a bottom, back of the lower end of the body 130′. The back strut 152 can include or be defined by an opening, cavity or web opening 152C.

The struts 150 can include a front strut 153, as shown in FIG. 3. The front strut 153 can be curved or straight and provided at or constitute a part of the bottom, front edge of the body 130′ of the lower cheek 130. The front strut 153 can include or be defined by an opening, cavity or web opening 153C.

Hereinafter, further features of the disclosure will be described.

The knee brace 10 with offset hinge 110, offset from the pivot point of the knee, can provide a separative force between the upper and lower leg. This separative force can increase as the leg extends, so as to absorb, at least in part, impact typically placed on the inner knee.

The knee brace can include an attachment mechanism configured to affix the brace to the leg above the knee. This can including rigid structure, fabric straps, cushion material, and rigid fasteners, for example.

The knee brace can include an attachment mechanism configured to affix the brace to the leg below the knee. This can including rigid structure, fabric straps, cushion material, and rigid fasteners, for example.

Attachment mechanisms, both above and below the knee, can allow the knee brace to be adjusted to fit a particular user, i.e. to fit a particular individual. The adjustment can be made to accommodate different body sizes and shapes. The adjustment can also be passive, without human intervention, such as by using elastic or compressive fabrics that form fit automatically.

The attachment mechanisms to provide for adjustment can also be active, such as a buckle. For example, the straps 300, including upper straps 310 and lower straps 320, as shown in FIG. 1 can include buckles interposed between sections of strap, so as to allow a user to adjust length and tightness of the strap around the user's leg. In some embodiments of the knee brace 10, the straps might be constructed of elastic material, such that buckles or other similar structure may not be needed. Illustratively, the straps of the embodiment of FIG. 1 may be constructed of elastic material, such that buckles may not be needed. VELCRO or other hook-and-loop fastener may also be used to attach different sections of strap together and/or to attach ends of strap together.

A knee brace of the disclosure can include a joint assembly, such as the offset hinge 110. The joint assembly, containing a rotation point, can connect the upper leg and lower leg attachment mechanisms. Such upper leg and lower leg attachment mechanisms can include the upper cheek 30 and the lower cheek 130, in accordance with at least one embodiment.

The embodiment of FIG. 1, for example, shows a knee brace 10 with a plurality of joints.

However, a knee brace of the disclosure is not limited to such particulars. A knee brace of the disclosure can include a single pivot point, a series of pivot points, or a deformation point that deflects out of a single piece.

The knee brace 10 with offset hinge 110, i.e. a joint assembly, can provide synchronized rotation, in that the knee brace 10 is configured to bend in unison with the knee. The joint assembly, of a knee brace of the disclosure, may either match or differ from the exact angle change of the knee.

An offset joint of a knee brace of the disclosure, such as offset hinge 110, can be a joint assembly that sits either in front of or behind the knee's natural pivot point.

The offset joint can be a cam, a single pivot point, or series of pivot points. This offset joint can be between 0.5 inches and 10 inches away from the knee's natural pivot point, or the center of the knee.

A knee brace of the disclosure, such as the knee brace 10, can provide length variation. Due

To construct of the offset joint, the knee brace can become longer relative to the leg when the leg is extended, than when the leg is contracted. Therefore, the knee brace is also shorter relative to the leg when the leg is contracted than when the leg is extended, due to a difference in the length of the lever arm created by the offset joint.

The knee brace of the disclosure can provide force generation. A difference in length based on leg extension creates a pushing force between the upper and lower leg. This leg extension can be driven by the natural power of the leg swinging forward, passive resistance, or an electronic aid.

The knee brace of the disclosure can provide impact reduction. That is, the force generation provided by the knee brace can reduce impact on the knee when landing on an extended leg, wherein the reduction may apply to both soft tissue and hard bone structure, and to either torn or intact tissue. This force generation may also provide general stability to weakened ligament tissue.

The knee brace of the disclosure can provide universal wearability. The design of the knee brace of the disclosure enables the knee brace to be worn by individuals with or without a medical condition. The knee brace of the disclosure can allow a user to perform activities that a user may nor otherwise be able to do.

The knee brace of the disclosure is reusable. The knee brace can be worn repeatedly, with materials that hold up to “wear and tear.” The knee brace of the disclosure can be put on and taken off by the end user.

In accordance with at least one embodiment of the disclosure, outlined below is an illustrative method for affixing a knee brace of the disclosure, such as the knee brace 10 of FIG. 1, to a user so as to achieve good fit and support. To begin, a user can sit with the user's leg bent and position the offset hinge in front of the kneecap. The user can secure a centrally located lower strap first (such as strap 322 shown in FIG. 1), wrapping such strap around the upper calf and fastening such strap with Velcro or a buckle, for example, for support below the knee. Then, the user can fasten an upper strap, e.g. strap 313 of FIG. 1, around the lower thigh, just above the knee, anchoring the brace in place on the user's leg. The user can then continue securing each strap in a pattern moving up and down the brace, adjusting each for even compression and comfort. For example, the user can alternate between securing and tightening one of the upper straps, then one of the lower straps, then one of the upper straps, and so forth. This approach to attaching the knee brace can ensure the hinge remains aligned in front of the knee, allowing the knee brace to track natural knee movement while providing stability. The straps of the knee brace may include and/or be fastened together using quick-release buckles, for example. Once the knee brace is initially fitted, the user can use such quick-release buckles, for example, to streamline putting the brace on and taking the brace off. Any arrangement, series or quantity of buckles, VELCRO, clips and clasps, for example, can be used to attach the knee brace to the user's leg and to resize the brace for the individual. Also, the straps of the knee brace can be constructed of any suitable material as desired, such as nylon and may be elastic to some degree.

The various components of embodiments of the disclosure may be made from any of a variety of materials including, for example, plastic, plastic resin, nylon, metal, aluminum, composite material, foam, rubber, wood, cloth, polycarbonate plastic, and/or ceramic, for example, or any other material as may be desired. For example, the device(s) of this disclosure may be produced from a plastic resin, such as polyethylene, and be injection molded.

A variety of production techniques may be used to make the apparatuses as described herein.

For example, suitable injection molding and other molding techniques and other manufacturing techniques might be utilized. Also, the various components of the apparatuses may be integrally formed, as may be desired, in particular when using molding construction techniques. Also, the various components of the apparatuses may be formed in pieces and connected together in some manner, such as with suitable adhesive and/or heat bonding.

The various apparatuses and components of the apparatuses, as described herein, may be provided in various sizes and/or dimensions, as desired.

It will be appreciated that features, elements and/or characteristics described with respect to one embodiment of the disclosure may be variously used and combined with other embodiments of the disclosure as may be desired.

In this disclosure, quotation marks, such as with “connection portion”, have been used to enhance readability and/or to parse out a term or phrase for clarity.

It will be appreciated that the effects of the present disclosure are not limited to the above-mentioned effects, and other effects, which are not mentioned herein, will be apparent to those in the art from the disclosure and accompanying claims.

Although the preferred embodiments of the present disclosure have been disclosed for illustrative purposes, those skilled in the art will appreciate that various modifications, additions and substitutions are possible, without departing from the scope and spirit of the disclosure and accompanying claims.

It will be understood that when an element or layer is referred to as being “on” another element or layer, the element or layer can be directly on another element or layer or intervening elements or layers. In contrast, when an element is referred to as being “directly on” another element or layer, there are no intervening elements or layers present.

It will be understood that when an element or layer is referred to as being “onto” another element or layer, the element or layer can be directly on another element or layer or intervening elements or layers. Examples include “attached onto”, secured onto”, and “provided onto”. In contrast, when an element is referred to as being “directly onto” another element or layer, there are no intervening elements or layers present. As used herein, “onto” and “on to” have been used interchangeably.

It will be understood that when an element or layer is referred to as being “attached to” another element or layer, the element or layer can be directly attached to the another element or layer or intervening elements or layers. In contrast, when an element is referred to as being “attached directly to” another element or layer, there are no intervening elements or layers present. It will be understood that such relationship also is to be understood with regard to: “secured to” versus “secured directly to”; “provided to” versus “provided directly to”; and similar language.

As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section could be termed a second element, component, region, layer or section without departing from the teachings of the present disclosure.

Spatially relative terms, such as “lower”, “lowest”, “top”, “upper”, “bottom”, “left”, “right” and the like, may be used herein for ease of description to describe the relationship of one element or feature to another element(s) or feature(s) as illustrated in the drawing figures. It will be understood that spatially relative terms are intended to encompass different orientations of structures in use or operation, in addition to the orientation depicted in the drawing figures. For example, if a device in the drawing Figs. is turned over, elements described as “lower” relative to other elements or features would then be oriented “lower” relative the other elements or features.

Thus, the exemplary term “lower” can encompass both an orientation of above and below. The device may be otherwise oriented (rotated 90 degrees or at other orientations) and the spatially relative descriptors used herein should be interpreted accordingly.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the disclosure are described herein with reference to diagrams and/or cross-section illustrations, for example, that are schematic illustrations of idealized embodiments (and intermediate structures) of the disclosure. As such, variations from the shapes of the illustrations as a result, for example, of manufacturing techniques and/or tolerances, are to be expected. Thus, embodiments of the disclosure should not be construed as limited to the particular shapes of components illustrated herein but are to include deviations in shapes that result, for example, from manufacturing.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Any reference in this specification to “one embodiment,” “an embodiment,” “example embodiment,” etc., means that a particular feature, structure, or characteristic described in connection with the embodiment is included in at least one embodiment of the disclosure. The appearances of such phrases in various places in the specification are not necessarily all referring to the same embodiment. Further, as otherwise noted herein, when a particular feature, structure, or characteristic is described in connection with any embodiment, it is submitted that it is within the purview of one skilled in the art to effect and/or use such feature, structure, or characteristic in connection with other ones of the embodiments.

Embodiments are also intended to include or otherwise cover methods of using and methods of manufacturing any or all of the elements disclosed above.

While the subject matter has been described in detail with reference to exemplary embodiments thereof, it will be apparent to one skilled in the art that various changes can be made, and equivalents employed, without departing from the scope of the disclosure.

All related art references discussed in the above Background section are hereby incorporated by reference in their entirety. All documents referenced herein are hereby incorporated by reference in their entirety.

In conclusion, it will be readily understood by those persons skilled in the art that the present disclosure is susceptible to broad utility and application. Many embodiments and adaptations of the present disclosure other than those herein described, as well as many variations, modifications and equivalent arrangements, will be apparent from or reasonably suggested by the present disclosure and foregoing description thereof, without departing from the substance or scope of the disclosure.

Accordingly, while the present disclosure has been described here in detail in relation to its exemplary embodiments, it is to be understood that this disclosure is only illustrative and exemplary of the present disclosure and is made to provide an enabling disclosure of the disclosure. Accordingly, the foregoing disclosure is not intended to be construed or to limit the present disclosure or otherwise to exclude any other such embodiments, adaptations, variations, modifications and equivalent arrangements.

What is claimed is:

1. A knee brace configured to be attached to a leg of a user, the knee brace comprising:
a left side assembly, the left side assembly including:
a left upper cheek configured to engage with an upper leg of the user;
a left lower cheek configured to engage with a lower leg of the user;
a left offset hinge that rotatably connects the left upper cheek to the left lower cheek, so as to be hinged about a first hinge axis;
a right side assembly, the right side assembly including:
a right upper cheek configured to engage with the upper leg of the user;
a right lower cheek configured to engage with the lower leg of the user;
a right offset hinge that rotatably connects the right upper cheek to the right lower cheek, so as to be hinged about the first hinge axis;
a first upper strap connected to the left upper cheek and the right upper cheek, the first upper strap configured to encircle the upper leg of the user;
a second upper strap connected to the left upper cheek and the right upper cheek, the second upper strap configured to encircle the upper leg of the user,
a third lower strap connected to the left lower cheek and the right lower cheek, the third lower strap configured to encircle the lower leg of the user;
a fourth lower strap connected to the left lower cheek and the right lower cheek, the fourth lower strap configured to encircle the lower leg of the user.

2. The knee brace of claim 1,
the left offset hinge includes at least one left plate, the lower end of the left upper cheek attached to the at least one left plate by a first pin; and the upper end of the left lower cheek attached to the at least one left plate by a second pin; and
the right offset hinge includes at least one right plate, the lower end of the right upper cheek attached to the at least one right plate by a third pin; and the upper end of the right lower cheek attached to the at least one right plate by a fourth pin.

3. The knee brace of claim 2,
the at least one left plate includes a pair of left plates, between which the left upper cheek and the left lower cheek are positioned, so as to be sandwiched; and
the at least one right plate includes a pair of right plates, between which the right upper cheek and the right lower cheek are positioned, so as to be sandwiched.

4. The knee brace of claim 3,
the lower end of the left upper cheek provided with a first gear rack; and
the upper end of the left lower cheek provided with a second gear rack, the second gear rack engaged with the first gear rack, so that rotation of the upper left cheek is mechanically tied to rotation of the lower left cheek.

5. The knee brace of claim 4,
the lower end of the right upper cheek provided with a third gear rack; and
the upper end of the right lower cheek provided with a fourth gear rack, the third gear rack engaged with the fourth gear rack, so that rotation of the upper right cheek is mechanically tied to rotation of the lower right cheek.

6. The knee brace of claim 2,
the lower end of the left upper cheek provided with a first gear rack; and
the upper end of the left lower cheek provided with a second gear rack, the second gear rack engaged with the first gear rack, so that rotation of the upper left cheek is mechanically tied to rotation of the lower left cheek; and
the lower end of the right upper cheek provided with a third gear rack; and
the upper end of the right lower cheek provided with a fourth gear rack, the third gear rack engaged with the fourth gear rack, so that rotation of the upper right cheek is mechanically tied to rotation of the lower right cheek.

7. The knee brace of claim 6, further including:
a first padding assembly attached to the first upper strap; and
a second padding assembly attached to the third lower strap.

8. The knee brace of claim 2,
the first pin including an internally threaded stand-off that receives a respective screw on both ends of the internally threaded stand-off;
the second pin including a second internally threaded stand-off that receives a respective screw on both ends of the internally threaded stand-off;
the third pin including a third internally threaded stand-off that receives a respective screw on both ends of the internally threaded stand-off; and
the fourth pin including a fourth internally threaded stand-off that receives a respective screw on both ends of the internally threaded stand-off.

9. The knee brace of claim 1,
the first upper strap connected to the left upper cheek and the right upper cheek, the first upper strap configured to encircle the upper leg of the user so as to have a first centroid defined by first material of the first upper strap, as such first material is positioned about the upper leg;
the second upper strap connected to the left upper cheek and the right upper cheek, the second upper strap configured to encircle the upper leg of the user so as to have a second centroid defined by second material of the second upper strap, as such second material is positioned about the upper leg, and
the first centroid and the second centroid defining an upper centroid line of attachment;
the third lower strap connected to the left lower cheek and the right lower cheek, the third lower strap configured to encircle the lower leg of the user so as to have a third centroid defined by third material of the third lower strap, as such third material is positioned about the lower leg;
the fourth lower strap connected to the left lower cheek and the right lower cheek, the fourth lower strap configured to encircle the lower leg of the user so as to have a fourth centroid defined by fourth material of the fourth lower strap, as such fourth material is positioned about the lower leg; and
the third centroid and the fourth centroid defining a lower centroid line of attachment, and
the upper centroid line of attachment and the lower centroid line of attachment being rotatable about a second hinge axis, and the second hinge axis being offset from the first hinge axis.

10. A knee brace configured to be attached to a leg of a user, the knee brace comprising:
a left side assembly, the left side assembly including:
a left upper cheek configured to engage with an upper leg of the user;
a left lower cheek configured to engage with a lower leg of the user;
a left offset hinge that rotatably connects the left upper cheek to the left lower cheek, so as to be hinged about a first hinge axis;
a right side assembly, the right side assembly including:
a right upper cheek configured to engage with the upper leg of the user;
a right lower cheek configured to engage with the lower leg of the user;
a right offset hinge that rotatably connects the right upper cheek to the right lower cheek, so as to be hinged about the first hinge axis;
a first upper strap connected to the left upper cheek and the right upper cheek, the first upper strap configured to encircle the upper leg of the user; and
a first lower strap connected to the left lower cheek and the right lower cheek, the third lower strap configured to encircle the lower leg of the user.
